(19) United States
(12) Patent Application Publication
(10) Pub. No.: US 2016/0100957 A1
(43) Pub. Date: Apr. 14, 2016

(54) ACETABULAR EXPLANT APPARATUS, SYSTEM, AND METHOD

(71) Applicant: Randall J. Lewis, Bethesda, MD (US)

(72) Inventor: Randall J. Lewis, Bethesda, MD (US)

(21) Appl. No.: 14/512,585

(22) Filed: Oct. 13, 2014

(51) Int. Cl. A61F 2/46; A61B 17/16

(52) U.S. Cl. CPC A61F 2/4609; A61B 17/1666; A61F 2002/4619

(57) ABSTRACT

An apparatus, system, and method for removing an acetabular cup from an acetabulum are provided herein. The apparatus includes a handle lever configured to extend from a shaft, the shaft having a longitudinal axis. A first impaction surface is disposed along the longitudinal shaft axis and a second impaction surface is offset from the longitudinal shaft axis. The apparatus includes a coupling portion at a proximal end of the shaft, a pivot element removably coupled to the coupling portion, the pivot element dimensioned to be received within an acetabular cup, and a blade removably coupled to the coupling portion and spaced from the pivot element, wherein when the pivot element is received within the acetabular cup, the blade is disposed externally of the acetabular cup adjacent an outer surface of the acetabular cup.

### BACKGROUND

Prosthetic ball-and-socket hip joints generally include a femoral component and an acetabular component. The acetabular component is a cup positioned in a patient's acetabulum, and serves as the socket of the hip joint. The acetabular cup is typically made of a metal material, such as titanium or cobalt chrome, having a porous hemispherical outer surface that allows the acetabular bone to grow in and anchor the cup within the acetabulum. Additionally, a liner of suitable material, such as ultra-high molecular weight polyethylene (UHMWPE), which is received within the acetabular cup, and includes a hemispherical recess for receiving the ball end of the femoral component of the prosthetic hip joint.

During a prosthetic hip joint revision procedure, the acetabular cup is removed from the acetabulum and replaced with a new acetabular cup. To remove the acetabular cup, a chisel-like osteotome is used, which includes a blade for cutting through the acetabular bone surrounding the hemispherical outer surface of the acetabular cup, and a head or anvil which may be struck by a mallet to drive the blade through the bone adjacent the acetabulum. In use, the blade of the osteotome is positioned near the acetabular cup, and the head of the osteotome is struck with the mallet to make a cut in the bone adjacent the acetabular cavity near the acetabular cup. Thereafter, the osteotome is withdrawn and repositioned, and the procedure is repeated until sufficient cuts have been made around the acetabular cup to permit removal thereof from the acetabulum. Thereafter, a new acetabular cup can be positioned within the acetabulum.

### SUMMARY

The present inventor has recognized, among other things, that what is needed is an improved apparatus and method for quickly and easily removing an acetabular cup from an acetabulum with minimal loss of acetabular bone, while preserving a relatively intact, hemispherical acetabular recess into which a new acetabular cup may be fitted. The present inventor has further recognized that superior force can be generated by striking a lever and/or a dedicated impaction surface connected to the lever to pivot the apparatus to cut around the acetabular cup in order to facilitate removal. The use of superior force can shorten surgery time and reduce anesthesia time and patient blood loss.

In one example disclosed herein, an apparatus for an acetabular explant procedure can include a shaft having a longitudinal shaft axis; a handle lever configured to extend from the shaft, the handle lever having a longitudinal lever axis; a first impaction surface disposed along the longitudinal shaft axis; a second impaction surface offset from the longitudinal shaft axis; a coupling portion at a proximal end of the shaft; a pivot element removably coupled to the coupling portion, the pivot element dimensioned to be received within an acetabular cup; and a blade removably coupled to the coupling portion and spaced from the pivot element, wherein when the pivot element is received within the acetabular cup, the blade is disposed externally of the acetabular cup adjacent an outer surface of the acetabular cup.

In another example disclosed herein, a system for removing an acetabular cup from an acetabulum can include an osteotome instrument, including; a handle lever having a longitudinal lever axis, a handle lever having a longitudinal lever axis coupled to the shaft at a first end, the handle lever having at least one impaction surface at or adjacent a second end thereof; a coupling portion at a proximal end of the shaft; a plurality of spherical head components each configured to be interchangeably and removably coupled to the coupling portion of the osteotome instrument; and a plurality of blades each configured to be interchangeably and removably coupled to the coupling portion of the osteotome instrument.

In another example disclose herein, a method for removing an acetabular cup from an acetabulum can include an osteotome instrument including a first impaction surface, a second impaction surface substantially orthogonal to the first impaction surface, a spherical head, and a blade spaced from the spherical head; seating the spherical head at least partially within the acetabular cup; and impacting the second impaction surface to move the osteotome instrument to make a series of cuts with the blade adjacent an outer surface of the acetabular cup.

In Example 20, the method of Example 19 optionally further comprises selecting the second impaction surface from a plurality impaction surfaces on a plurality of extensions, the plurality of extensions having one or more different sizes and/or shapes; and coupling the second impaction surface to the osteotome instrument.

In Example 21, the implant or method of any one or any combination of Examples 1-20 is optionally configured such that all elements or options recited are available to use or select from.

These and other examples and features of the present prosthetic implants and methods will be set forth in part in the following Detailed Description. This Summary is intended to provide non-limiting examples of the present subject matter—it is not intended to provide an exclusive or exhaustive explanation. The Detailed Description below is included to provide further information about the present tapered adapter and methods.

### BRIEF DESCRIPTION OF THE DRAWINGS

In the drawings, which are not necessarily drawn to scale, like numerals may describe similar components in different views. Letters may represent distances or dimensions. The drawings illustrate generally, by way of example, but not by way of limitation, various embodiments discussed in the present document.

FIG. 1 illustrates a perspective view of a plurality of components of an acetabular explant system, including an osteotome instrument, an interchangeable spherical head, a pair of interchangeable blades, a torque wrench, and an adaptor, in accordance with at least one example of the present disclosure;

FIG. 2 illustrates a perspective view of an osteotome instrument, an impactor tool, and an acetabulum of a hip joint including an acetabular cup fitted therein, in accordance with at least one example of the present disclosure;

FIG. 2A illustrates an enlarged top view of a portion of the osteotome instrument comprising a handle lever and an impaction surface, in accordance with at least one example of the present disclosure;

FIG. 3 illustrates an enlarged top view of another example of a portion of the handle lever and the impaction surface, in accordance with at least one example of the present disclosure;

FIG. 4 illustrates a partial section view of a spherical head of an osteotome instrument seated within an acetabular cup liner prior to cutting with an osteotome having a blade, in accordance with at least one example of the present disclosure;

FIG. 5 illustrates a partial section view of the osteotome instrument of FIG. 4 in a pivoted position with a first osteotome blade cutting bone adjacent the acetabulum to a first depth, in accordance with at least one example of the present disclosure;

FIG. 6 illustrates a partial section view of the osteotome instrument of FIG. 4 with a second osteotome blade cutting bone adjacent the acetabulum to a second depth, in accordance with at least one example of the present disclosure;

FIG. 7 illustrates a front perspective view of an interchangeable osteotome having a long blade for use with an osteotome instrument, in accordance with at least one example of the present disclosure;

FIG. 8 illustrates a rear perspective view of the interchangeable osteotome in FIG. 7, in accordance with at least one example of the present disclosure; and

FIG. 9 illustrates a flow diagram for a method of removing an acetabular cup, in accordance with at least one example of the present disclosure.

### DETAILED DESCRIPTION

The present disclosure relates generally to an apparatus, system, and method for removing an acetabular cup from an acetabulum. The apparatus includes a handle lever having a longitudinal lever axis coupled to a shaft, the shaft having a longitudinal shaft axis. A first impaction surface is located generally adjacent the longitudinal shaft axis and a second impaction surface is offset from the longitudinal shaft axis. In some instances, the second impaction surface is disposed so as to be substantially orthogonal to the first impaction surface. The can include a coupling portion coupled to a proximal end of the shaft, a spherical pivot element removably coupled to the coupling portion, the pivot element dimensioned to be received within an acetabular cup, and a blade removably coupled to the coupling portion and spaced from the pivot element, wherein when the pivot element is received within the acetabular cup, the blade is disposed externally of the acetabular cup adjacent an outer surface of the acetabular cup.

The configuration of the apparatus with the second impaction surface offset from the longitudinal shaft axis allows a torque to be generated, for example, by impacting the second impaction surface with an impactor tool, such as a mallet. Larger degrees of torque can be generated by striking the second impaction surface with the impactor tool than could otherwise be generated by applying manual force by hand to the handle lever. The torque that results from impacting the second impaction surface can rotate the shaft substantially about the longitudinal shaft axis and can allow the blade to cut around the acetabular cup more efficiently with reduced effort. This helps to minimize procedure time, physician fatigue, and patient blood loss.

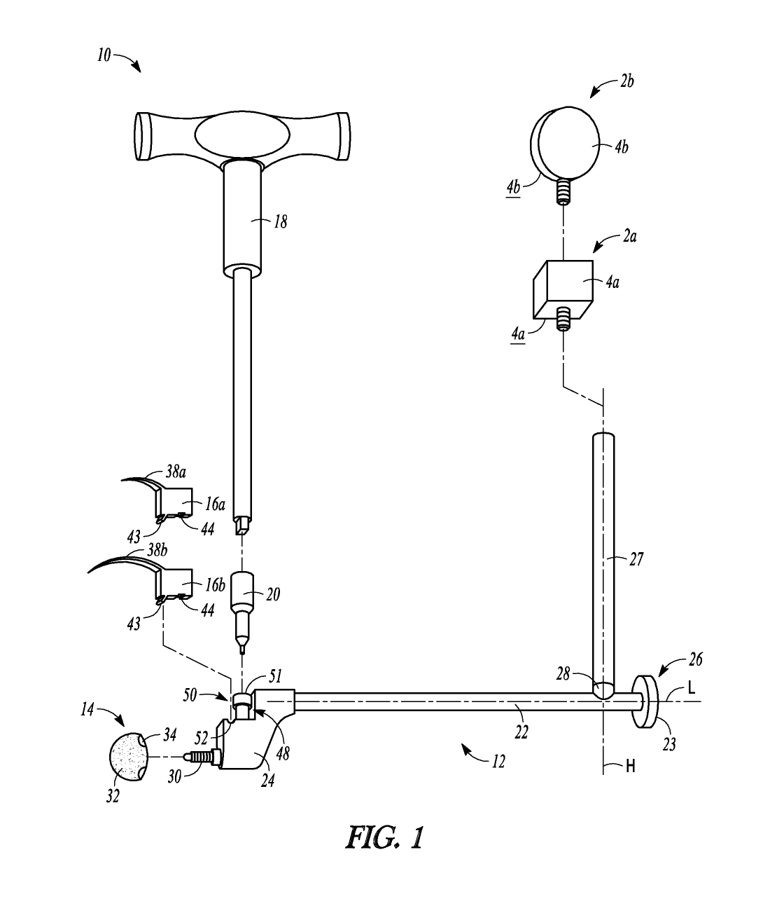

Referring to FIG. 1, surgical system 10 is shown, which can generally include an osteotome instrument 12, one of a plurality of interchangeable pivot elements such as spherical heads 14, a plurality of interchangeable osteotomes 16a, 16b, a driver tool 18, and a coupling shaft 30 for interchangeably securing the osteotomes 16a, 16b to the osteotome instrument 12. Although only one spherical head 14 and two osteotomes 16a, 16b are depicted any number of spherical heads or osteotomes can be included in the surgical system 10. The osteotome instrument 12 can include a shaft 22 including a coupling portion 24 at a proximal end thereof. A handle lever 27 can be coupled at a coupling location 28 of the shaft 22. The shaft 22 can include a longitudinal shaft axis L, and the handle lever 27 can include a longitudinal lever axis H. The longitudinal lever axis H can intersect the longitudinal shaft axis L at any suitable angle, such as a right angle as illustrated in FIG. 1. As described herein, the coupling location 28 can include, for example, a threaded connection, a snap fit connection, a press fit connection, or a fixed connection, such as a welded coupling. In an example, the handle lever 27 and shaft 22 can be formed as a single unitary object. In an example, the shaft 22 and handle lever 27 can be grasped to manipulate the osteotome instrument 12, as described herein. In another example, the shaft 22 and the handle lever 27 can be coupled so as to be perpendicular with respect to one another.

The osteotome instrument 12 can include a first impaction surface 26 along the longitudinal shaft axis L of the shaft 22. For example, the impaction surface 26 can be positioned at a distal end of the shaft 22. The impaction surface 26, in an example, can be a top surface 23 of an expanded portion of the shaft 22. The expanded portion can provide a benefit, including, but not limited to, a larger surface area for impaction by a mallet (not shown) or other suitable striking instrument. The osteotome instrument 12 can include a unitary or removable extension, such as extensions 2a and 2b in FIG. 1, located at an end of the handle lever 27 opposite of the coupling location 28. Extensions 2a and 2b can each include one or more second impaction surfaces 4a and 4b, respectively. In an example, each of the extensions 2a and 2b can include a pair of substantially opposing impaction surfaces that can allow the lever 27 to rotate in a first direction about the longitudinal shaft axis L by impacting one of the pair of impaction surfaces, and to rotate in an opposite, second direction about the longitudinal shaft axis L by impacting the other one of the pair of impaction surfaces. Although a pair of substantially opposing impaction surfaces is described, any number of impaction surfaces can be provided without departing from the intended scope of the present disclosure. As set forth above, the second impaction surfaces 4a and 4b can be permanently coupled to the handle lever 27, or alternatively, the second impaction surfaces 4a and 4b can be provided on modular extensions 2a and 2b that can be coupled to the handle lever 27 as desired. The particular configuration of the impaction surfaces 4a and 4b can be selected as desired by the surgeon. In some instances, it may not be necessary or desirable to utilize the second impaction surfaces 4a and/or 4b. In such circumstances, the surgeon may instead contact one or more surfaces of the handle lever 27 directly with the impactor tool. As will be discussed in further detail below, the handle lever 27 can be provided with one or more flat or substantially flat surfaces for this purpose. However, handle levers 27 having any shape and exterior contour can be contacted with an impactor tool to generate a torque for cutting around an acetabular cup, including a handle lever 27 having a generally circular cross-section.

As described herein, the second impaction surfaces 4a and/or 4b can provide one or more benefits, including but not limited to, easier more rapid removal of an acetabular cup from an acetabulum by providing greater torque on the handle lever 27 of the osteotome instrument 12 compared to manually pulling or pushing the handle lever 27. Additionally, the second impaction surfaces 4a and/or 4b can help facilitate easier more rapid removal of the implant by allowing high impact forces to be used in cutting the acetabular cup circumferentially from the hip. As can be appreciated, cutting through acetabular bone and other tissues can be very difficult unless a source of external torque greater than that provided by a surgeon manually pulling or pushing the handle lever 27 by hand is provided as described herein.

The coupling portion 24 of the osteotome instrument 12 can include a coupling shaft 30 projecting therefrom generally parallel to shaft 22, onto which the spherical head 14 can be attached to secure the spherical head 14 to the osteotome instrument 12. Any suitable means of attachment can be used including, but not limited to, threads, a press-fit connection, a snap-fit connection, or the like. In an example, the coupling shaft 30 can have external threads configured to engage with internal threads in the spherical head 14. The spherical head 14 can include a substantially spherical pivot surface 32, such as at a bottom of the spherical head 14. In an example, the spherical head 14 can include a plurality of indentations 34 therein to allow spherical head 14 to be easily grasped during attachment of the spherical head 14 to the coupling shaft 30, or detachment therefrom. The surgical system 10 can include a plurality of spherical heads 14 of different sizes, each spherical head 14 sized for use with acetabular cups of different sizes. Suitable spherical heads 14 and osteotomes 16a, 16b can be selected depending upon the dimensions of the acetabular cup to be removed such that, when a corresponding spherical head 14 and osteotome 16a, 16b are attached to the instrument, the spacing between the blade 38a, 38b and center of the spherical head 14 corresponds to the spacing between the center of the recess in the acetabular cup liner and the rim of the acetabular cup. In an example, a plurality of osteotomes 16a, 16b can be provided, wherein each osteotome includes a blade, such as 38a, 38b. Each of the plurality of osteotomes 16a, 16b can be dimensioned to correspond to a corresponding acetabular cup. The system 10 can include the plurality of blades 38a, 38b wherein each blade of the plurality of blades has different dimensions, such as a blade length. That is, the system 10 can include corresponding sets of osteotomes 16a, 16b, blades 38a, 38b, and spherical heads 14.

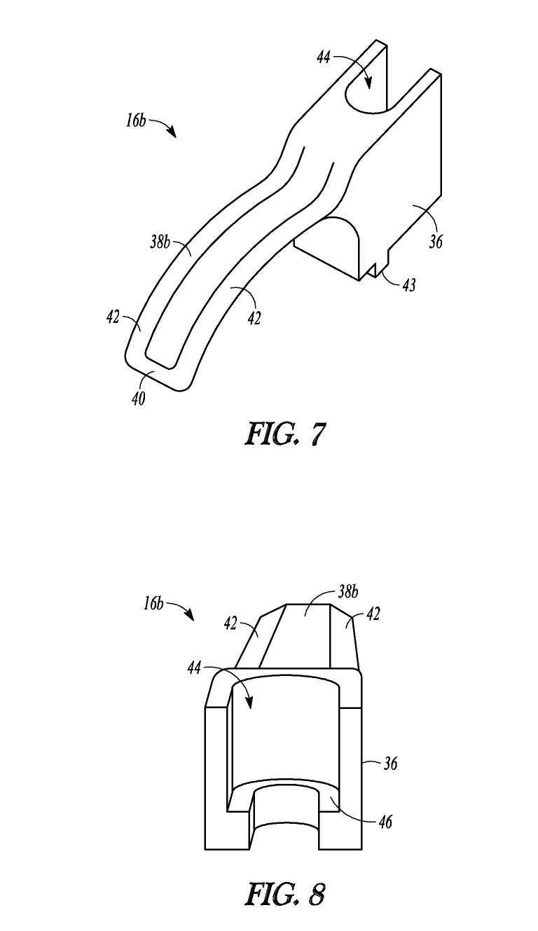

Referring again to FIG. 1, the osteotome instrument 12 can further include an osteotome-receiving feature or recess 48 in the coupling portion 24 thereof, having a notch 52. An osteotome clamping screw 50 can be disposed within the recess 48 and threaded into (or otherwise coupled to) the coupling portion 24 of the osteotome instrument 12. The osteotome clamping screw 50, in an example, can be movable between a loosened and a tightened position, and further, may be retained by the coupling portion 24 in the loosened position to prevent detachment of the osteotome clamping screw 50, so as to simplify the interchanging of osteotomes 16a, 16b during a surgical procedure. As an example, the osteotome 16b is described in more detail in reference to FIGS. 7 and 8 herein.

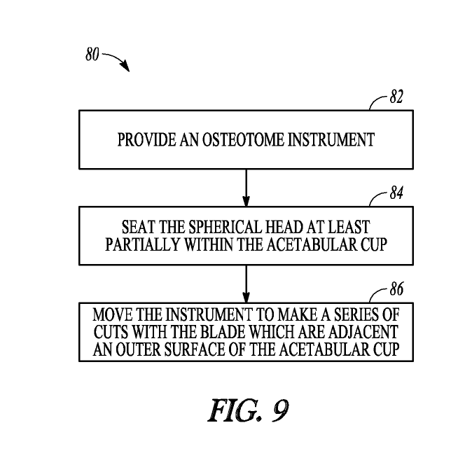

A shown in FIG. 1, to attach one of the osteotomes 16a, 16b to the instrument 12, the chosen osteotome can be placed within the recess 48 such that the osteotome clamping screw 50 is disposed within the slot 44 of the blade 38a, 38b, and a tooth 43 of the blade 16a, 16b is fitted within the notch 52 with the blade clamping screw 50 in a loosened position. Thereafter, the osteotome clamping screw 50 can be tightened using the driver tool 18 and adaptor 20 such that a collar 51 of osteotome clamping screw 50 engages a ledge 46 (see FIG. 9) of the blade 16a, 16b to secure the blade. The driver tool 18 (see FIG. 1) can be adjusted to deliver a predetermined amount of torque to the osteotome clamping screw 50, so as to prevent the collar 51 of osteotome clamping screw 50 from being tightened onto the ledge 46 of the osteotome 16a, 16b beyond a desired clamping force.

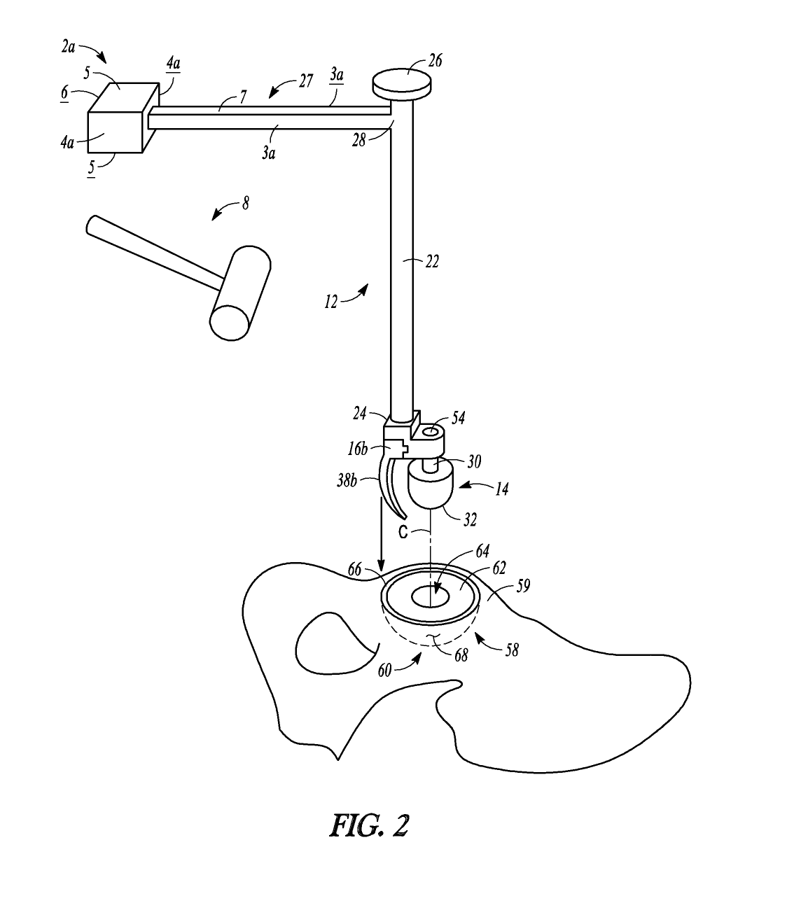

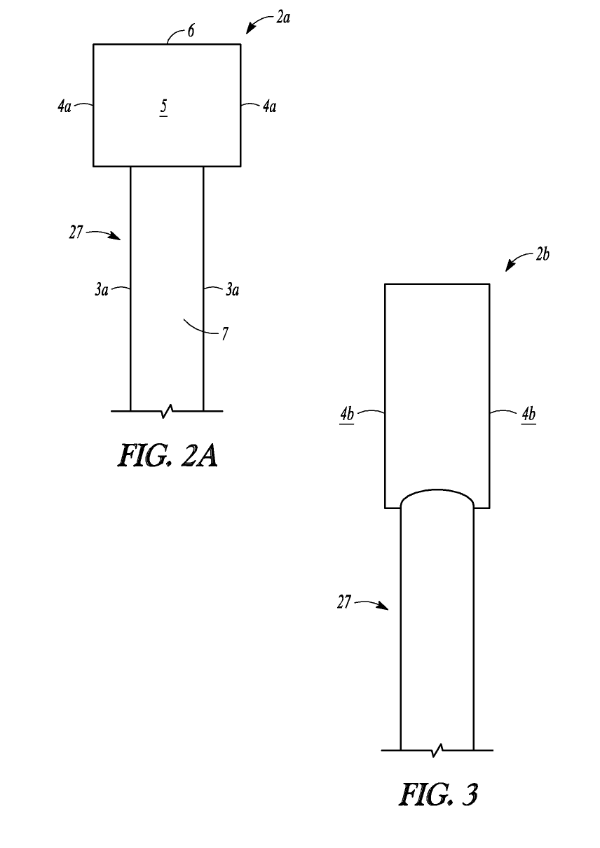

FIG. 2 illustrates a perspective view of the osteotome instrument 12, a impactor tool 8 and an acetabulum 58 of a hip joint including an acetabular cup 60 fitted therein, in accordance with at least one example of the present disclosure. As described herein, the second osteotome 16b having a longer blade 38b can be attached to the instrument, as described in relation to FIG. 1. The spherical head 14, selected from a plurality of spherical heads, can be seated within the hemispherical recess 64 of a cup liner 62 of the acetabular cup 60, which is anchored in the acetabulum 58. As shown in FIG. 2, the handle lever 27 can include any suitable cross-sectional shape or combination of shapes, such as circular, square, or rectangular, for example. FIG. 2 depicts the handle lever 27 having a rectangular or square cross-section with substantially flat first and second side surfaces 3a. These side surfaces 3a can be impacted by an impaction tool in a manner similar to impaction surfaces 4a in order to generate a larger torque transfer to the shaft 22, which, in turn, can be transferred to the cutting surfaces of the blade 38b to allow for quicker, easier, and more effective cutting of the acetabulum 59 and surrounding tissues.

In an example, the handle lever 27 can include the extension 2a at an opposite end of the handle lever 27 from the coupling location 28. The extension 2a can include the second impaction surfaces 4a (side surfaces of the extension 2a), top and underside surfaces 5, and a front surface 6. If necessary, each surface of the extension 2a can be used as a striking surface so as to aid in cutting the bone 59 adjacent the acetabular cup 60, as described herein. As illustrated, the pair of impaction surfaces 4a of the extension 2a can be utilized in some examples to aid in providing a dedicated impact area for the impactor tool. In other examples, a dedicated impactor surface may not be utilized and contact can be made with the substantially flat first and/or second side surfaces 3a and 3b. One or both of the second impaction surface(s) 4a and the extension 2a can be offset longitudinally from the first impaction surface 26 (e.g., a top surface) of the handle lever 27. In some cases, the second impaction surface(s) 4a can be disposed substantially orthogonal to the first impaction surface 26. This arrangement can facilitate rotation of the shaft 22 about the longitudinal shaft axis (FIG. 1) by striking one of the second impaction surface(s) 4a, as well as facilitating translation of the shaft 22 axially along the longitudinal shaft axis by striking the first impaction surface 26. In some instances the first impaction surface 26 can provide the benefit of an area for an operator to grab while striking the second impaction surface(s) 4a with a mallet or other suitable impaction instrument.

Impactor tool 8 can apply a significant amount of impact force to the second impaction surface(s) 4a (and/or first and second side surfaces 3a and 3b) as well as the first impaction surface 26. This force is magnified by the length of the handle lever 27 to facilitate torque about the shaft 22. This torque can cause rotation of components attached to the shaft 22 such as the osteotome 16b and blade 38b. The torque causes the blade 38a to cut around the acetabular cup 60 as previously described.

FIG. 2A illustrates a top view of a portion of the handle lever 27 and the extension 2a, in accordance with at least one example of the present disclosure. The extension 2a can include a pair of opposing second impaction surfaces 4a. The handle lever 27 can include the first and second side surfaces 3a and 3b previously described. Furthermore, the extension 2a can include the top 5 surface, underside surface (not shown), and the front surface 6.

As shown in FIG. 2A, the first and second side surfaces 3a and 3b of the handle lever 27, the top and bottom surfaces 5 of the extension 2a, the top surface 7 of the handle lever 27, and the second impaction surface(s) 4a of the extension 2a can be substantially flat (i.e. have a small amount of curvature). However, in some examples the second impaction surface(s) 4a (and the first and second side surfaces 3a and 3b, the top surface 7, etc.) can be provided with curvature as desired. Substantially flat surfaces allow the various impaction surfaces to be more effectively contacted by the impactor tool 8 (FIG. 2) to provide force to cut around the acetabular cup 60.

FIG. 3 illustrates a top view of a portion of the handle lever 27 and the alternative extension 2b, in accordance with at least one example of the present disclosure. The extension 2b can include at least one or, as shown, a pair of opposing second impaction surfaces 4b. As is illustrated in FIG. 3, the extension 2b can be cylindrical in shape with the second impact surfaces 4b comprising both ends thereof. As previously illustrated the extension 2b (and extension 2a, etc.) can be a modular component that can be selected and attached to the handle lever 27 according to some examples. In other examples, the extension 2b (and extension 2a, etc.) can be formed integral with the handle lever 27. In yet other examples, each handle lever 27 can be formed with a unique extension, and the handle lever 27 can be removable coupled to the shaft 22.

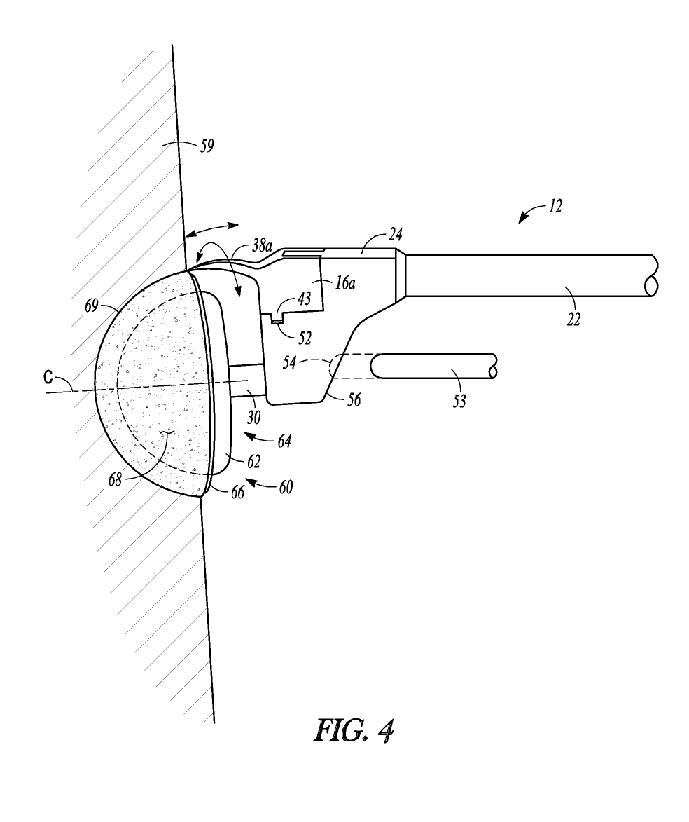

FIGS. 4-6 illustrate an exemplary use of the osteotome instrument 12 to remove an acetabular cup 60, in accordance with at least one example of the present disclosure. The first osteotome 16a having the first blade 38a, such as a short blade, can be attached to the osteotome instrument 12, as described herein in relation to FIGS. 1 and 7-8. The spherical head 14 of the osteotome instrument 12 can be seated within a hemispherical recess 64 of the liner 62 of the acetabular cup 60, which is anchored in the acetabulum 58 of the patient. An impactor tool 53 can be pressed against the dimple 54 in a rear side 56 of the coupling portion 24 so as to aid in seating the spherical head 14 within the recess 64 of the acetabular cup 60. For example, the impactor tool 53 can include an elongated rod member dimensioned to be at least partially received by the dimple 54. Although shown as being positioned only slightly below a surface on the rear side 56, the dimple 54 can, in an example, extend further within the coupling portion 24, such as ¼, ½, ¾, or more within the rear side 56.

As shown in the example illustrated in FIG. 4, the first blade 38a of the osteotome 16a can be spaced a distance from the center of the spherical head 14 which corresponds to the distance between a center C of recess 64, or the center of spherical head 14, and a rim 66 of the acetabular cup 60, such that the first blade 38a is disposed closely adjacent an outer hemispherical surface 68 of the acetabular cup 60.

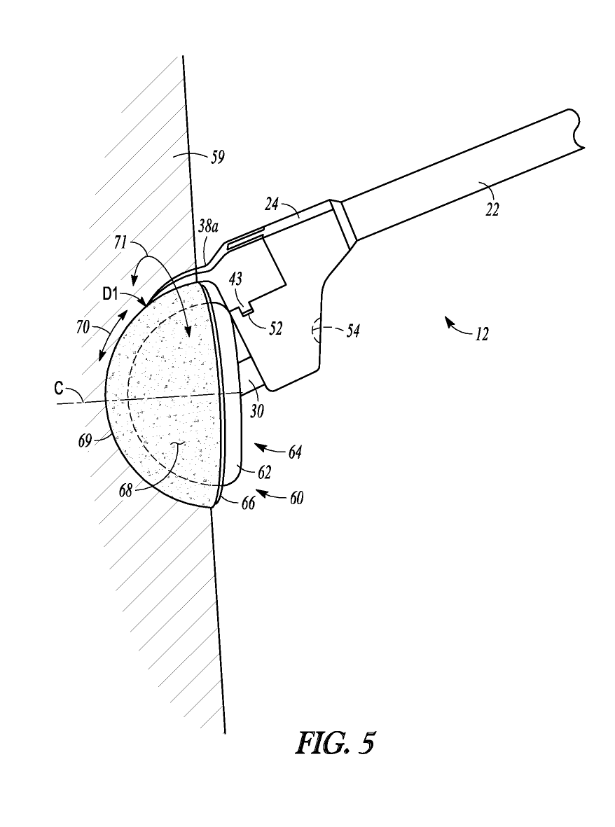

As illustrated in FIG. 5, the first impaction surface can be stuck to drive the first blade 38a into the bone 59 as indicated by arrow 70. The first blade 38a can include an end cutting edge that facilitates cutting along path 70. The shaft 22 of the osteotome instrument 12 can be pivoted by, for example, striking one or more of the second impaction surface(s) (see 4a, 4b in FIGS. 2-3) and/or side surface (see 3a, 3b in FIGS. 2-2A) such that as the first blade 38a moves along the path of arrow 71 as shown in FIG. 5. A side cutting edge(s) of the first blade 38a cuts bone 59 around the outer hemispherical surface 68 of the acetabular cup 60 to a first depth D1. The first depth D1 extends substantially from the rim 66 to an apex 69 of the acetabular cup 60. Thereafter, the shaft 22 of the osteotome instrument 12 can be pivoted in the reverse direction along the path of the arrow 70 to withdraw the first blade 38a. During such withdrawal, the engagement between the tooth 43 of the osteotome 16a within the notch 52 in the coupling portion 24 of the osteotome instrument 12 can prevents disengagement of the osteotome 16a from the instrument 12 in the event that the blade clamping screw 50 is not sufficiently tightened.

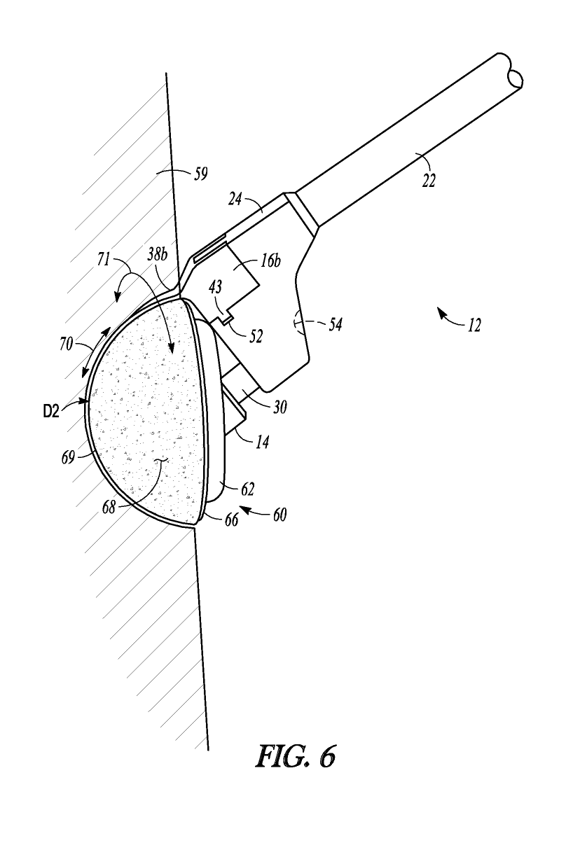

The offset location of the top surface 5 of extension 2a (FIGS. 2 and 2A) relative to the first impaction surface 26 can provide for an angled driving force imparted on the blade 38a. In an example, the underside surface 5 of the extension 2a can be struck to adjust the blade 38a relative to the bone 59. As discussed, a large amount of impact force can be applied to the handle lever 27 (FIGS. 1 and 2) via the second impaction surface 4a, 4b, and/or side surfaces 3a, 3b, etc. This force is magnified by the length of the handle lever 27 to create a large torque used to rotate the osteotome instrument 12 such that the blade 38a cuts around the acetabular cup 60. Thus, as shown by arrow 71 in FIG. 5, the handle lever 27 of the osteotome instrument 12 can be used to rotate the first blade 38a, striking at least one second impaction surface (see 4a, 4b in FIGS. 2-3) and/or side surface of the handle lever 27 (see 3a, 3b in FIGS. 2-2A) as necessary with a mallet or other striking instrument, to make an orbital cut about the outer hemispherical surface 68 of the acetabular cup 60 using the first blade 38a. Advantageously, as can be seen in FIGS. 5 and 6, the first blade 38a is spaced closely adjacent the outer hemispherical surface 68 of acetabular cup 60 during the cutting procedure described herein, allowing bone 59 around the acetabular cup 60 to be cut closely adjacent to acetabular cup 60, such that loss of bone 59 is minimized.

As shown in FIG. 6, the second osteotome 16b having the second blade 38b, such as a long blade, may be attached to the coupling portion 24 of the instrument 12 as described above, wherein the second blade 38b extends to the apex 69 of the acetabular cup 60 when the osteotome instrument 12 is actuated along the direction of the arrow 70. The osteotome instrument 12, with the osteotome 16b attached thereto, can be used in the same manner as described above with respect to the osteotome 16a to cut bone 59 surrounding the periphery of the acetabular cup 60 from the first depth D1, to a second depth D2 at apex 69 of the acetabular cup 60. After the bone 59 surrounding the acetabular cup 60 is cut away therefrom following path indicated by arrow 71, the acetabular cup 60 can be removed from the acetabulum 58, leaving an intact, hemispherical recess in the acetabulum 58 into which a replacement acetabular cup may be fitted. In an example, the top surface 5 of the extension 2a (FIGS. 2 and 2a) can be struck downwardly to aid in removing the acetabular cup 60, such as by providing torque due to the offset location of the top surface 5 relative to the longitudinal shaft axis of the shaft 22.

FIGS. 7 and 8 illustrate perspective views of an interchangeable osteotome, such as the osteotome 16b, in accordance with at least one example of the present disclosure. As discussed above, the osteotomes 16a, 16b may be interchangeably connected to coupling portion 24 of instrument 12. The osteotome 16b is shown in FIGS. 7 and 8 for purposes of example, and can include head portion 36 with curved blade 38b extending therefrom. The blade 38b can include an end cutting surface 40 and a pair of side cutting surfaces 42. The head portion 36 of the osteotome 16b can also include a tooth 43, and a slot 44 having a semi-annular ledge 46.

FIG. 9 illustrates a flow diagram of a method 80 for removing an acetabular cup. An osteotome instrument can be provided or obtained at 82. The osteotome instrument can be, for example, the osteotome instrument 12 described herein. The osteotome instrument can include a first impaction surface, a second impaction surface offset from the first impaction surface, a spherical head, and a blade spaced from the spherical head. In an example, the spherical head can be fully received in the acetabular cup 84. The method 80 can include selecting a spherical head from a plurality of spherical heads of different sizes and coupling the spherical head to the osteotome instrument, as described herein.

At 86, the instrument can be moved to make a series of cuts with the blade which are adjacent to an outer surface of the acetabular cup. For example, the handle lever of the osteotome instrument can be pivoted, such as by striking the second impaction surface with a mallet or similar instrument, or by striking one of the side surfaces of the handle lever. In an example, the method 80 can include selecting a blade from a plurality of blades of different dimensions and coupling the blade to the instrument, as described herein. In some examples a surface of the handle lever, an extension of the handle lever, or another surface can be impacted so as to remove the acetabular cup from the acetabulum. Impacting, for example, an extension of the handle lever or the handle lever itself can provide an upward (lift) moment to the osteotome and the blade, such that the acetabular cup can be removed or otherwise forced out of the acetabulum, after the acetabular cup has been freed, by cutting of the adjacent bone.

To better illustrate the acetabular explant apparatus and related methods disclosed herein, a non-limiting list of examples is provided here:

In Example 1, an apparatus for an acetabular explant procedure can include a shaft having a longitudinal shaft axis; a handle lever configured to extend from the shaft, the handle lever having a longitudinal lever axis; a first impaction surface disposed along the longitudinal shaft axis; a second impaction surface offset from the longitudinal shaft axis; a coupling portion at a proximal end of the shaft; a pivot element removably coupled to the coupling portion, the pivot element dimensioned to be received within an acetabular cup; and a blade removably coupled to the coupling portion and spaced from the pivot element, wherein when the pivot element is received within the acetabular cup, the blade is disposed externally of the acetabular cup adjacent an outer surface of the acetabular cup.

In Example 2, the apparatus of Example 1 is optionally configured such that wherein the handle lever is threadably coupled to the shaft.

In Example 3, the apparatus of any one or any combination of Examples 1-2 is optionally configured such that wherein at least a portion of a length of the handle lever has a square or rectangular cross-section.

In Example 4, the apparatus of any one or any combination of Examples 1-3 is optionally configured such that wherein the handle lever is coupled to the shaft at a first end, and wherein the second impaction surface is located at an opposing second end of the handle lever.

In Example 5, the apparatus of any one or any combination of Examples 1-4 is optionally configured to further comprise an extension on the second end of the handle lever, the extension including the second impaction surface.

In Example 6, the apparatus of any one or any combination of Examples 1-5 is optionally configured such that the extension is removably coupled to the handle lever.

In Example 7, the apparatus of any one or any combination of Examples 1-6 is optionally configured to further comprise a plurality of interchangeable and removable extensions each capable of being coupled to the second end of the handle lever.

In Example 8, the apparatus of any one or any combination of Examples 1-7 is optionally configured such that the second impaction surface is substantially flat or slightly rounded and can serve as an impaction surface substantially perpendicular to the first impaction surface.

In Example 9, the apparatus of any one or any combination of Examples 1-8 is optionally configured such the second impaction surface is configured to facilitate rotation of the handle lever about the longitudinal lever axis, and wherein the first impaction surface is configured to facilitate axial movement of the handle lever when the pivot element is received in the acetabular cup.

In Example 10, the apparatus of any one or any combination of Examples 1-9 is optionally configured such that the second impaction surface is substantially orthogonal to the first impaction surface.

In Example 11, the apparatus of any one or any combination of Examples 1-10 is optionally configured such that the second impaction surface comprises two opposing surfaces to allow for impaction in a first direction and/or a second reverse direction.

In Example 12, the apparatus any one or any combination of Examples 1-11 is optionally configured such that the first impaction surface is configured to facilitate driving the blade into a bone adjacent the acetabular cup and the second impaction surface is configured to facilitate cutting around the acetabular cup.

In Example 13, a system for removing an acetabular cup from an acetabulum can include an osteotome instrument, including; a handle lever having a longitudinal lever axis, a handle lever having a longitudinal lever axis coupled to the shaft at a first end, the handle lever having at least one impaction surface at or adjacent a second end thereof; a coupling portion at a proximal end of the shaft; a plurality of spherical head components each configured to be interchangeably and removably coupled to the coupling portion of the osteotome instrument; and a plurality of blades each configured to be interchangeably and removably coupled to the coupling portion of the osteotome instrument.

In Example 14, the system of Example 13 is optionally configured such that the impaction surface comprises two opposing surfaces to allow for impaction in a first direction and/or a second direction.

In Example 15, the system of any one or any combination of Examples 13-14 is optionally configured such that the handle lever has an extension at the second end thereof, and wherein the extension including the second impaction surface.

In Example 16, the system of any one or any combination of Examples 13-15 is optionally configured such that the extension comprises a plurality of extensions each configured to be interchangeably and removably coupled to the handle lever.

In Example 17, the system of any one or any combination of Examples 13-16 is optionally configured such that the impaction surface is arranged on the handle lever and spaced from the shaft to facilitate rotation of the shaft about the longitudinal shaft axis when the second impaction surface is struck.

In Example 18, the system of any one or any combination of Examples 12-17 is optionally configured such that the impaction surface comprises one or more side surfaces of the handle lever.

In Example 19, a method for removing an acetabular cup from an acetabulum can include an osteotome instrument including a first impaction surface, a second impaction surface substantially orthogonal to the first impaction surface, a spherical head, and a blade spaced from the spherical head; impacting the first impaction surface to seat the spherical head at least partially within the acetabular cup; and impacting the second impaction surface to move the osteotome instrument to make a series of cuts with the blade adjacent an outer surface of the acetabular cup.

In Example 20, the method of Example 19 optionally further comprises selecting the second impaction surface from a plurality impaction surfaces on a plurality of extensions, the plurality of extensions having one or more different sizes and/or shapes; and coupling the second impaction surface to the osteotome instrument.

In Example 21, the implant or method of any one or any combination of Examples 1-20 is optionally configured such that all elements or options recited are available to use or select from.

The above Detailed Description includes references to the accompanying drawings, which form a part of the Detailed Description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. As used herein, the term “about” when used in connection with a range or a value of dimension indicate within machine or manufacturing tolerance ranges.

The above Detailed Description is intended to be illustrative, and not restrictive. For example, the above-described examples (or one or more aspects thereof) may be used in combination with each other. Other examples can be used, such as by one of ordinary skill in the art upon reviewing the above Detailed Description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

What is claimed is:

1. An apparatus for an acetabular explant procedure, comprising:
a shaft having a longitudinal shaft axis;
a handle lever configured to extend from the shaft, the handle lever having a longitudinal lever axis;
a first impaction surface disposed along the longitudinal shaft axis;
a second impaction surface offset from the longitudinal shaft axis;
a coupling portion at a proximal end of the shaft;
a pivot element removably coupled to the coupling portion, the pivot element dimensioned to be received within an acetabular cup; and
a blade removably coupled to the coupling portion and spaced from the pivot element, wherein when the pivot element is received within the acetabular cup, the blade is disposed externally of the acetabular cup adjacent an outer surface of the acetabular cup.

2. The apparatus of claim 1, wherein the handle lever is threadably coupled to the shaft.

3. The apparatus of claim 1, wherein at least a portion of a length of the handle lever has a square or rectangular cross-section.

4. The apparatus of claim 1, wherein the handle lever is coupled to the shaft at a first end, and wherein the second impaction surface is located at an opposing second end of the handle lever.

5. The apparatus of claim 4, further comprising an extension on the second end of the handle lever, the extension including the second impaction surface.

6. The apparatus of claim 5, wherein the extension is removably coupled to the handle lever.

7. The apparatus of claim 4, further comprising a plurality of interchangeable and removable extensions each capable of being coupled to the second end of the handle lever.

8. The apparatus of claim 1, wherein the second impaction surface is substantially flat.

9. The apparatus of claim 1, wherein the second impaction surface is configured to facilitate rotation of the handle lever about the longitudinal lever axis, and wherein the first impaction surface is configured to facilitate axial movement of the handle lever when the pivot element is disposed in the acetabular cup.

10. The apparatus of claim 1, wherein the second impaction surface is substantially orthogonal to the first impaction surface.

11. The apparatus of claim 1, wherein the second impaction surface comprises two opposing surfaces to allow for impaction in a first direction and/or a second reverse direction.

12. The apparatus of claim 1, wherein the first impaction surface is configured to facilitate driving the blade into a bone adjacent the acetabular cup and the second impaction surface is configured to facilitate cutting around the acetabular cup.

13. A system for removing an acetabular cup from an acetabulum, comprising:
an osteotome instrument, including;
a shaft having a longitudinal shaft axis;
a handle lever having a longitudinal lever axis coupled to the shaft at a first end, the handle lever having at least one impaction surface at or adjacent a second end thereof;
a coupling portion coupled to a proximal end of the shaft;
a plurality of spherical head components each configured to be interchangeably and removably coupled to the coupling portion of the osteotome instrument; and
a plurality of blades each configured to be interchangeably and removably coupled to the coupling portion of the osteotome instrument.

14. The system of claim 13, wherein the impaction surface comprises two opposing surfaces to allow for impaction in a first direction and/or a second direction.

15. The system of claim 13, wherein the handle lever has an extension at the second end thereof, and wherein the extension including the second impaction surface.

16. The system of claim 15, wherein the extension comprises a plurality of extensions each configured to be interchangeably and removably coupled to the handle lever.

17. The system of claim 13, wherein the second impaction surface is arranged on the handle lever and spaced from the shaft to facilitate rotation of the shaft about the longitudinal shaft axis when the second impaction surface is struck.

18. The system of claim 12, wherein the impaction surface comprises one or more side surfaces of the handle lever.

19. A method for removing an acetabular cup from an acetabulum comprising:
providing an osteotome instrument including a first impaction surface, a second impaction surface substantially orthogonal to the first impaction surface, a spherical head, and a blade spaced from the spherical head;
seating the spherical head at least partially within the acetabular cup; and
impacting the second impaction surface to move the osteotome instrument to make a series of cuts with the blade about an outer surface of the acetabular cup.

20. The method of claim 18, further comprising:
selecting the second impaction surface from a plurality impaction surfaces on a plurality of extensions, the plurality of extensions having one or more different sizes and/or shapes; and
coupling the second impaction surface to the osteotome instrument.
